(19) United States
(12) Plant Patent Application Publication
(10) Pub. No.: US 2010/0333244 P1
(43) Pub. Date: Dec. 30, 2010

(54) Lemna minor plant named 'Henry Blanke'

(50) ;

(75) Inventors: Maria Rogmans, Kalkar (DE); Hermann-Josef Wilhelm, Kalkar (DE)

Correspondence Address: BUCHANAN, INGERSOLL & ROONEY PC, POST OFFICE BOX 1404, ALEXANDRIA, VA, 22313-1404, US

(73) Assignee: Maria Rogmans, Kalkar (DE)

(21) Appl. No.: 12/801,389

(22) Filed: Jun. 7, 2010

(30) Foreign Application Priority Data
Jun. 5, 2009 (EP) 2009/0984

(51) Int. Cl. A01H 5/00

(52) U.S. Cl. PLT/342

(57) ABSTRACT

A new Lemna minor plant named ‘Henry Blanke’ is provided. The plant is particularly well suited to serve as a biomass renewable energy source. The plant is small in stature but unlike Dwarf Duckweed possesses short roots which well enable the absorption of nutrients present in water. Such short roots also enable the ready separation of adjoining plants. Strong green leaves are displayed. The plant is well able to maintain active biosynthesis under low light conditions. The plant is particularly well suited to metabolize carbon dioxide from emissions with a simultaneous increase of mass.

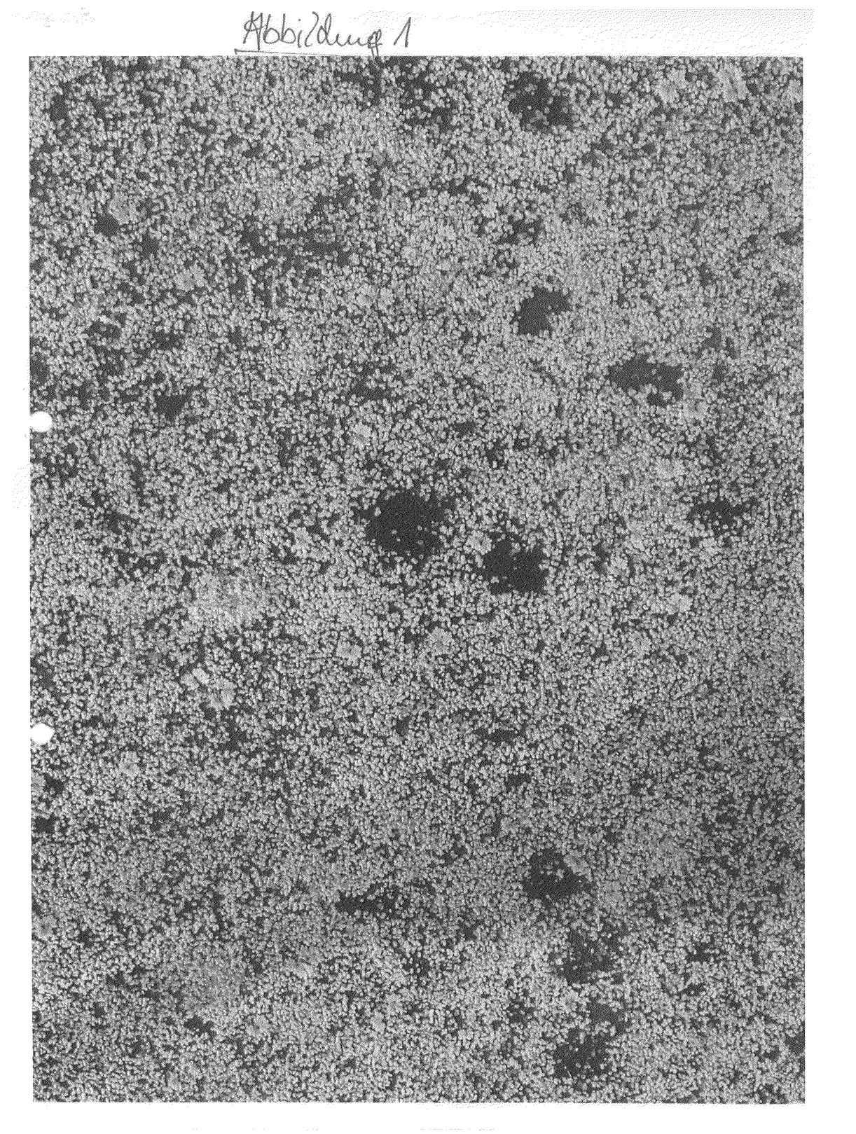

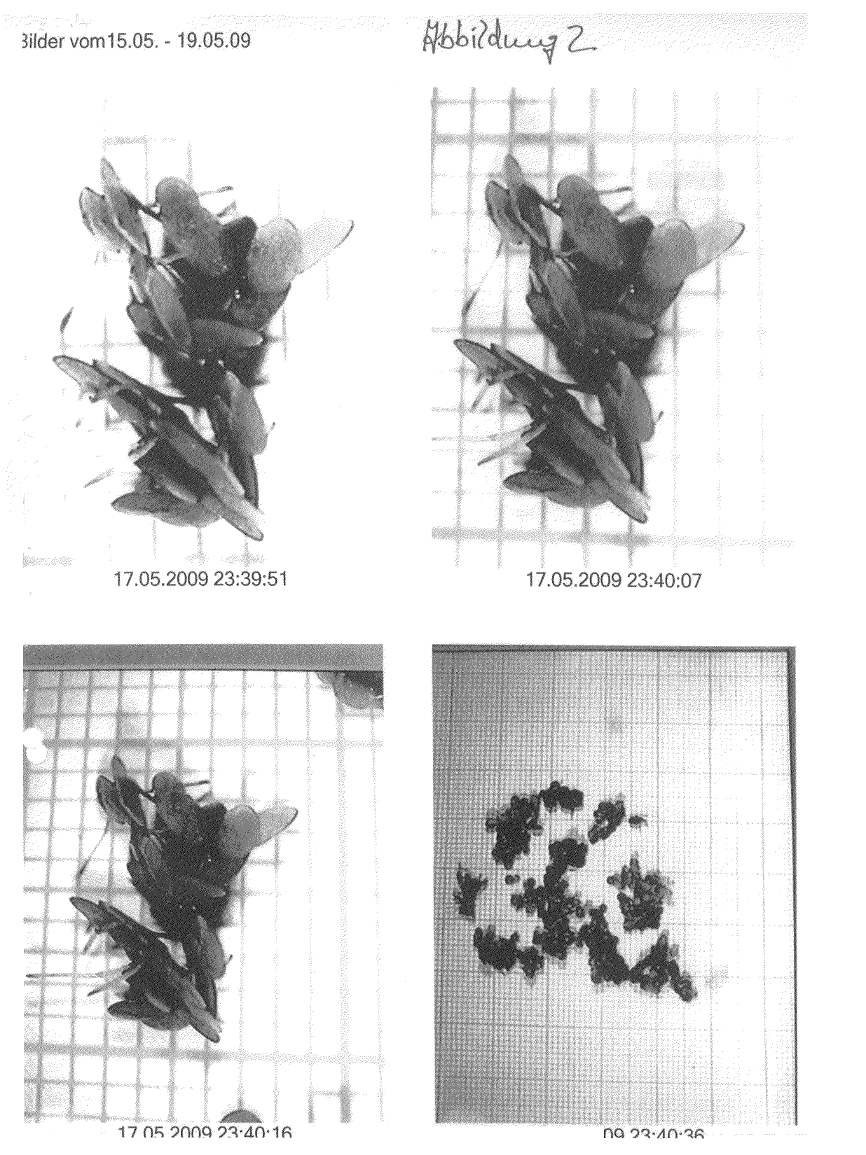

The invention relates to Lemnacea, especially the taxonom Lemnacea minor L.

Lemna minor L are well known under the trivial name duckweed. Their natural environment are small lakes with soft water.

The common object for all underspecies was, to breed a family of varieties, with a little structure (because of higher mathematical propagation: the more individuals you have per volumina, the more potential propagation takes place, the higher is the result of biomass), and they all have to produce high biomass under only very FEW LIGHT.

So all subspecies are able to produce carbon-hydrates by MIXOTHROPHY. (ENERGY Input not only by photosynthesis, but also biochemical energy input as well as warmness)

The first basical variety is Lemnacea minor L ‘Henry Blanke’.

### DESCRIPTION OF VARIETY

### BOTANICAL TAXON

### GENUS

LEMNA

### SPECIES

LEMNA MINOR

### COMPARISON SPECIES

Lemna Minor L

### NEW VARIETY

Variety denomination: Lemna Minor Henry Blanke

### BREEDING PROCESS

The new variety has been selected by meticulous selection from a reproduction tank with a large number of different interbreeding Lemna. From the naturally provoked crossings, the crossings which showed a maximum green saturation, thus evidencing the most effective metabolism, were selected. Subsequent laboratory studies revealed that it was exactly these new Lemna which showed an increased CO₂ metabolism. Afterwards, more selecting and separating was performed, and an isolated reproduction process started from this point.

Because known Lemna are not homogeneous in their colouring and change between green and greenish brown, the selection of automatically interbreeding new varieties was performed according to the following three criteria:
small root, which allows easy separation of plants
noticeable extremely small size
preferably, a homogeneous strong green colouring.

The motivation for the choice of these three criteria for selection was the objective to generate a Lemna by selective breeding, which is extremely small in order to achieve a clearly better mathematical precondition for a high mass potentiation, and which, in addition, has a strong, chlorophyll-active, preferably homogeneous green colour, which is an indicator for an optimized CO₂ metabolism. Further, it was taken care that the new Lemna does not remain completely rootless, as is the dwarf duckweed Wolffia arrhiza. The latter is admittedly smaller than the new variety of Lemna, but it does not have any roots at all, which results in the fact that it has difficulty absorbing mineral nutrients from the water. In contrast, this had to be avoided in the new variety of Lemna, because due to the intended use the ability to absorb mineral nutrients plays a key role for the cascade use referred to below.

Consequently, the strong, chlorophyll-containing, green colour was one of the breeding criteria in order to maintain active photosynthesis processes also in the presence of low light. Thus the quality to tolerate low light conditions was even more distinctive in the new variety. For technical application, the new variety was intended to be more easily separable than the comparison variety despite its reduced—but still present—root.

### MORPHOLOGICAL DESCRIPTION

Form, size, colour: oval to round, 1.5 to 2 mm, upper and lower side of the leaves RHS dark green (41) 144 A to RHS dark green (41) 141 A.

Photo 1 (Henry Blanke) shows a total population of the candidate variety in a surprising homogeneity. The forms and colourings are as described above.

Photo 2 (Henry Blanke) shows, on the one hand, that the candidate variety is even smaller than the comparison variety. On the other hand, it shows much more clearly that the roots of the candidate variety HENRY BLANKE are visibly shorter than the ones in the original plant. Due to the visibly shorter roots, the Lemna are much better to be separated, and they clump together less. As an additional result, there was a stronger potentiation in their reproduction.

### DISTINCTIVE FEATURES WITH RESPECT TO THE COMPARISON VARIETY

Size: In contrast to the comparison variety, the new variety is remarkably smaller.
Roots: only a comparably short root at the lower side of the leaf in contrast to the comparison variety, which possesses relatively long roots and tends to clump together. The new variety does only minimally clump together or not at all due to its shorter roots. This is an important separation aspect for technical application. In the new variety, roots are present but clearly shorter than in the comparison variety. This means that the new variety still has roots in complete contrast to the rootless known dwarf duckweed, which is completely rootless.
Readiness to grow: The readiness to grow is remarkable. The smallness of the new variety has the advantage that, according to the mathematical propagation principle of biomass,

Delta n(t)=c*n(t)
it offers a larger initial number NO per weight unit. This results in a larger lever in the potentiation of the reproduction. The result is a maximum yield of biomass within short reproduction times
Supplemental breeding scheme: The Lemna were induced to produce isolated, barely visible flowers under the stimulation of light. The induced natural readiness to breed by this allowed the observation of morphological variations. A low light phase (50 to 100 Lux) followed. This means that the Lemna population was maintained at this low level of light over several days and weeks, so that only the Lemna which were able to reproduce and to assert themselves under these conditions were selected. With the specific selection parameters for low light conditions, those Lemna were developed from a larger number of Lemna, which showed a maximum green saturation (chlorophyll), because only those were capable of carrying out metabolic processes under such low light conditions.
Metabolic activity: The new variety has a larger effective (photosynthesis and metabolically active) surface per volume due to its remarkable smallness. Thus, in relation to the total biomass, there results a ratio of gas exchange rate to biomass, which is clearly larger than the one in the comparison variety. As Lemna is generally attributed to a high tolerance of high CO₂ concentrations, this feature has still been increased in the new variety because of the biophysical correlation described above. Thus the new variety is particularly suited to effectively metabolize CO₂ from emissions with a simultaneous increase of mass. Due to the spectrum of ingredients, the biomass can be used for what is referred to as cascade application, which means:
1^{st} step: binding of CO₂ as biocatalyst
2^{nd} step: extraction of chemical ingredients
3^{rd} step: energetic exploitation of residue
4^{th} step: recycling of CO₂ resulting from the energetic exploitation in 1^{st} step.
Further observation: In the new variety HENRY BLANKE, which has been selected for low light tolerance, it was reproducibly observed that it automatically moves with a speed of 30 to 50 cm per hour from a heavily illuminated area to the shadowed area. The related mechanisms are based on the fact that the plant, and particularly its root, is capable of moving along the gradient of the temperature-dependent surface tension. We are currently in the process of study whether there are any additional biochemical or biophysical effects.

1. (canceled)

2. A new and distinct Lemna minor plant, substantially as illustrated and described.
